(19) United States
(12) Patent Application Publication
(10) Pub. No.: US 2007/0110683 A1
(43) Pub. Date: May 17, 2007

(54) Oral care formulation

(75) Inventors: Jonathan B. Levine, Purchase, NY (US); Gary A. Durga, Marlboro, NJ (US)

Correspondence Address: H. JAY SPIEGEL - H. JAY SPIEGEL & ASSOCIATES, P.O. BOX 11, MOUNT VERNON, VA, 22121, US

(73) Assignee: GoSMILE, INC.

(21) Appl. No.: 11/585,115

(22) Filed: Oct. 24, 2006

Related U.S. Application Data
(60) Provisional application No. 60/736,836, filed on Nov. 16, 2005.

(51) Int. Cl. A61K 8/97; A61K 38/43; A61K 36/82

(52) U.S. Cl. 424/58; 424/729; 424/769; 424/94.1

(57) ABSTRACT

An oral care formulation comprises disclosed ranges of proportions of Pycnogenol, Uncaria Tomentosa Extract, CoQ10, Stabilized Vitamin C, Green Tea Extract, flavorant, sweetener and water. The inventive formulation is applied within the oral cavity using any suitable applicator. The formulation is operative to reduce bad breath, gingivitis, gingival bleeding, plaque build-up and inflammation of soft and hard tissues of the oral cavity. Other constituent ingredients, as disclosed, may also be included in the inventive formulation for the described purposes.

The present invention relates to an oral care formulation. Applicant claims priority from Provisional Application Ser. No. 60/736,836, filed Nov. 16, 2005.

### BACKGROUND OF THE INVENTION

There are over 500 species of bacteria living in the mouth at any given time. These bacteria constantly form dental plaque (the biofilm) which is a sticky colorless film that adheres to one's teeth and can cause health problems. Lack of brushing and flossing regularly to keep the teeth clean causes the plaque to build up along the gumline, creating an environment for greater colonization of the bacteria between the teeth and under the gums.

Americans brush on average for 38 seconds instead of the recommended 2 minutes twice a day, and only 10% of Americans floss regularly, resulting in 70% of Americans over the age of 35 having some form of gum disease and bad breath (Oral Malodor). Bad breath is composed of sulfur based compounds and can have an odor or smell such as that of “rotten eggs.” As a result, bad breath is perceived by the generic public as offensive, especially during a conversation among friends or during intimate occasions such as kissing or, in general, whenever someone opens their mouth. As a result, bad breath has become a popular concern of the generic public resulting in the need for other forms of oral care products such as mouthwashes and/or breath sprays to control this offensive odor.

Unfortunately, many products currently sold do not work very well and only mask the problem for a short amount of time without getting to the true etiology of the offensive odors. Additionally, free radicals are harmful compounds or molecules that cause cellular damage to the body. A “free radical” is an atom that has lost an electron and is an unstable, high energy fragment that can catalyze other free radicals by absorbing energy and electrons from other molecules. This process can cause cellular damage to the body if the electrons and energy are not replenished and the free radical is not “de-activated.” Scientists estimate that each cell in our body (billions of cells) suffers 10,000 free radical hits each day. The amount of damage that results depends upon how well the cells are protected by molecules and atoms that are antioxidants or replenish the lost electron.

Aging is the process whereby the number of healthy cells in the body is reduced. The body responds to a challenge and stays in a state called homeostasis. When this state is altered, and the body loses this ability, the system breaks down. For example, the mass of healthy active cells in each organ declines as a person ages, thus the organ's function is diminished. The cumulative effect of billions of cellular free-radical reactions is the loss of cells. This can happen at the cell membrane level (transport nutrients into the cell and export waste products out); when cell DNA is damaged and won't function properly, there is lysosomal damage (release of harmful toxins in the cell), as well as when proteins fuse together and do not function properly. The result of many of these free radical reactions is that the number of active cells in the body decreases; causing aging and other health related issues.

The present invention can confer protection to specific areas from aging or also referred to as oxidative stress. To qualify as an antioxidant, a few good molecules must protect many, many other molecules by neutralizing bad molecules or fragments of molecules. Our bodies generate some antioxidants and we are dependent on the diet to supply many antioxidants. Important antioxidant nutrients come from vitamins, minerals, amino acids, natural extracts, herbal supplements and coenzymes.

A number of antioxidants regenerate other antioxidant molecules. For example, Pycnogenol can regenerate “used” or “spent” vitamin C, which in turn can regenerate used vitamin E. This means that Pycnogenol enables the sparse amounts of vitamin C and vitamin E found in most diets to function as if they were higher in the diet. The mechanism is such that when vitamin E or vitamin C molecules come in contact with free radicals, they donate an electron to the free radical, making it a normal non-reactive molecule. At this point, when the inactive vitamin C or E comes in contact with Pycnogenol, it can be regenerated back into active vitamin C. Active vitamin C can also regenerate an inactive vitamin E radical. When the powerful antioxidant complexes are working together, the recycling and replacement effect can return the inactive molecules into their protective antioxidant forms.

There is a growing body of evidence that shows a link between the chronic inflammation of gum disease and the affect this has on the heart and the circulatory system, causing an increase of atherosclerosis and stroke. A study published in the Journal of Circulation (February, 2005) found that people with gum disease had a higher incidence of carotid artery plaque, an indicator of cardiovascular disease. This leads to the supposition that atherosclerosis (plaque buildup in the innermost layer of the artery walls) may be reduced or even reversed by simply reducing the level of disease-causing periodontal bacteria.

By focusing on the health of the oral cavity through powerful antioxidants and vitamins, overall systemic condition can be impacted. In the inventive formulation, there are a number of actives which can have a direct impact in reducing atherosclerotic plaque formation and other inflammatory diseases that occur in the body such as chronic bowel syndrome, diabetes, arthritis via a direct impact on the oxidative effect of free radicals, antibacterial properties, restoring damaged cells and reducing plaque and gingivitis. This formulation, in its preferred embodiments, can be in several forms such as liquid, solid or semi-solid, can be taken orally, can be readily absorbed or the rate of dissolution can be modified to be released at certain time points in the gastrointestinal tract.

Of the countless bacteria living inside our mouths, some are essential for providing the first line of defense against infection while others, in excess, wreak havoc, causing everything from bad breath to plaque and tartar buildup. These phenomena can result in inflammation, infection or, in the worst case scenario, tooth loss. These types of “bad” bacteria can be absorbed in the bloodstream, impacting a number of chronic inflammatory diseases such as cardiovascular disease and diabetes, and perhaps contributing to conditions resulting in low birthrate babies.

Ironically, conventional approaches to battling halitosis may be doing more harm than good. This is because many mouthwashes contain a significant percentage of alcohol as a constituent ingredient. The alcohol is evaporative in nature and creates a drier, more acidic environment inside the mouth, thereby fostering a breeding ground for anaerobic bacteria, the microbes that are responsible for bad breath. This delicate balance of acid and base that is normally modulated by our protective saliva is thrown off due to the alcohol effect on the mucosa and the decreased levels of saliva. Additionally, high levels of alcohol, which most mouthwashes contain, combined with smoking has been linked to throat cancer. By having a more balanced pH, moisturizing environment in one's mouth, a healthier bacterial balance would result. This may be accomplished through a comprehensive approach that combines proper oral hygiene practices and nutrient intake supplemented with the active ingredients of the present invention. An approach including use of the inventive oral care formulation would enhance the health of the mouth and, thereby, of the body as a whole.

Applicant is aware of the following published U.S. patent applications:
US 2002/0146463 A1 to Clayton
US 2003/0044359 A1 to Wuelknitz et al.
US 2004/0039078 A1 to Suh et al.
US 2005/0069501 A1 to Ibrahim et al.
US 2005/0100537 A1 to Evans et al.
US 2005/0288387 A1 to Feng et al.

Applicant is aware of the following U.S. Patent:
U.S. Pat. No. 6,500,408 to Chen.

Each of the above-listed documents teaches a composition including one or more of the constituent ingredients of the present invention. However, none of them teaches the entire combination of constituent ingredients set forth herein nor do any of them teach the features, aspects and purposes of the present invention. The present invention has been clinically tested to show its benefits and purposes.

### SUMMARY OF THE INVENTION

The present invention relates to a new oral care composition or formulation sold under the Trademark SMILECEUTICALS which contains AC-11, Pycnogenol (bioflavanoid) CoQ10, vitamin C, and vitamin E, in amounts from 0.0001 to 35%, by weight, and other ingredients, dissolved in a suitable vehicle for application into the oral cavity to improve the health of the gum tissues, oral mucosa and to prevent gum disease and halitosis.

The mouth has extremely vascular tissue with a high number of blood vessels. Proper circulation is necessary, and this tissue has a high metabolism, requiring a great amount of energy. The active ingredients in the SMILECEUTICAL formulation directly affect ATP support since CoQ10 stimulates the production of ATP in the mitochondria. The Pycnogenol strengthens and seals fragile capillaries of the gum, reduces plaque accumulation on the teeth, vitamin C prevents gum inflammation and has an antibacterial effect, and AC-11 improves the ability to repair DNA from oxidative stress, toxins and pollution.

All of these active ingredients are antioxidants that have the ability to scavenge free radicals and otherwise reduce the quantity or negative effects of reactive oxygen (free radicals). When taken together, a replenishing effect occurs with Pycnogenol and vitamin C and an additive effect occurs when all of the active ingredients are formulated together.

Whereas a single antioxidant compound such as vitamin E and vitamin C is protective against several specific kinds of free radicals, a mixture of many different types of antioxidants protects against a greater range of free radicals. The antioxidant ability to protect cell membranes, cell interiors (mitochondria) and DNA repair creates a unique and powerful team of antioxidants. Many of the antioxidants can regenerate themselves. Pycnogenol can regenerate “used” or “spent” vitamin C, which in turn can regenerate vitamin E. This means that Pycnogenol enables sparse amounts of vitamin C and vitamin E found in most diets to function as if they were in higher amounts in their diet.

The SMILECEUTICALS formulation is a focused approach of vitamins for the oral cavity that promotes health locally and has local uptake that promotes systemic health.

The activity of the SMILECEUTICALS composition is connected with the health of the oral cavity. Applicant's formulation has the ability to repair DNA from oxidative stresses, bind and repair capillaries to improve circulation, increase ATP formation in mitochondria to create improved energy in the cells, and neutralize the oxidative stresses due to aging, environmental pollutants, inflammation, cancer, alcohol damage, and radiation injury. The therapeutic action of the SMILECEUTICALS composition is clearly evident in reduction in inflamed and impaired tissues such as gingival inflammation around the teeth and the mucosal membranes in the oral cavity.

The typical route of administration for active oral care ingredients is through a pill or some type of supplement that has to go through the gastrointestinal tract, delaying action and having a lesser chance of achieving the desired effect. In addition, in contrast to the typical route of administration, the present invention also utilizes transmucosal uptake via the mouth which is fast, readily absorbed, and immediately goes to work without going through the gastrointestinal tract.

The dosages described are of concentrations that have a direct oral cavity effect and a systemic effect for overall wellness. The SMILECEUTICALS composition needs to be delivered in such a way that is easy and convenient for a person to use and can be in a liquid, solid or semi-solid form. Some examples of the delivery systems are: a spray, a rinse, a chewing gum, a mint, a floss, a lip balm, a toothpaste, a nylon tip, and a powdered or tablet supplement.

The present invention describes a composition for the oral care industry. However, it can be extended to the food, pharmaceutical, personal care and confectionary industry. This composition along with proper solubilizing agents can offer protection against oxidative stresses due to aging, environmental pollution, cancer, alcohol damage, radiation injury/therapy, and can create improved energy in the cells, repair and regenerate damaged cells and neutralize free radicals.

The composition according to the present invention may also contain other active ingredients in addition to the principle (SMILECEUTICALS) actives. Examples of other active ingredients are Fluoride compounds, Triclosan, Stannous compounds, Zinc compounds, etc.

The inventive compositions are specifically designed to help a person have healthy gum tissues, even though only 10% of the population in the United States floss their teeth regularly. People who do not floss their teeth often have some sort of gum inflammation.

Accordingly, it is a first object of the present invention to provide an oral care formulation.

It is a further object of the present invention to provide such a formulation that includes a prescribed proportional combination of Pycnogenol, AC11, CoQ10, and certain vitamins.

It is a still further object of the present invention to provide such a formulation wherein the vitamins are vitamins A, C and E, and the formulation also includes aloe vera, allantoin and can contain vitamins D and B complex.

It is a still further object of the present invention to provide such a formulation including cooling properties and breath freshening properties through inclusion of ingredients such as essential oils, of which some examples are eucalyptus, thymol, and menthol.

It is a still further object of the present invention to provide such a formulation with antioxidant properties as well as the ability to retard gum inflammation and to reduce the presence of “bad” bacteria.

It is a yet further object of the present invention to provide such antioxidants which have the ability to protect cell membranes and cell interiors while regenerating themselves and others of the constituent ingredients.

These and other objects, aspects and features of the present invention will be better understood from the following detailed description of the preferred embodiments when read in conjunction with the appended drawing figures.

### BRIEF DESCRIPTION OF THE DRAWINGS

FIG. 1 shows a bar graph of mean organoleptic judge scores for product A and the placebo comparing the base line values with six weeks hence.

FIG. 2a shows a bar graph of oral chroma in ng/10 ml for product A and the placebo comparing the base line values with six weeks hence.

FIG. 2b is similar to FIG. 2a, but measures the values in ppb.

FIG. 3 shows a bar graph of indices of plaque and gingiva for product A and the placebo comparing the base line values with six weeks hence.

FIG. 4 shows a bar graph of the % bleeding on probing for product A and the placebo comparing the base line values with six weeks hence.

### SPECIFIC DESCRIPTION OF THE PREFERRED EMBODIMENTS

In the preferred embodiment of the present invention, the constituent ingredients of the inventive formulation are the following:

| Ingredient | Approximate w/w % |
|---|---|
| Pycnogenol | 0.01-15% |
| ACll | 0.01-15% |
| CoQI0 | 0.01-15% |
| Stabilized Vitamin C | 0.01-15% |
| Green Tea Extract | 0-15% |
| Flavor | 0.01-20% |
| Sweetener | 0.01-10.0% |
| Water | qs |

Preservatives such as Sodium Benzoate, Sorbate, Sorbic Acid, Propyl and/or Methyl Parabens can also be added as appropriate.

These ingredients are included in the inventive formulation for the following reasons:
1. Pycnogenol is a super-antioxidant 100 times more effective against free radical damage than vitamins A, C and E, and 50 times as potent as Coenzyme Q10 (CoQ10). Pycnogenol helps prevent gum disease by reducing plaque buildup, decreasing inflammation, improving circulation, minimizing gingival bleeding and providing mild anti-fungal and antibacterial support.
2. AC-11 has been shown to convert DNA damaged by oxidative stress into healthy DNA, and provides anti-aging benefits by promoting cellular rejuvenation.
3. CoQ10 is a potent antioxidant complex comprised of a combination of B-complex vitamins and vitamin C. CoQ10 is commonly used in skin care as part of an anti-aging regimen. In an ingestible form, it neutralizes free radicals which can damage healthy cells. The damaged cells are those implicated in periodontal disease, heart disease and cancer, and helps convert food into energy, providing the body with the necessary fuel for optimal functioning.
4. Stabilized vitamin C {in conjunction with vitamins A & E} is universally recognized for immune enhancement. Vitamin C helps keep the soft tissue in the mouth healthy, making it more resistant to bacterial infection associated with gingivitis. It also helps suppress inflammation, thus lowering the risk of periodontal infection.
5. Aloe Vera promotes healing while providing anti-inflammatory benefits. It also has antiseptic properties, to help protect against gum disease.
6. Allantoin is derived from wheat germ, rice polishings and comfrey roots, and helps promote healing and stimulates healthy tissue formation.
7. Essential oils of eucalyptus, thymol and menthol provide cooling properties and help to freshen breath and kill “bad” bacteria.
8. Green Tea Extract, when included, is an antioxidant that works solely or synergistically with the other active ingredients and delivers anti-microbial and anti-bacterial properties to relieve gingivitis, plaque build-up and bad breath. It also helps to protect oral soft tissue from harmful bacteria and free radicals.

Applicant has found that the combination of constituent ingredients listed above results in synergistic activity between complementary antioxidants. For example, Pycnogenol can regenerate vitamin C which in turn can regenerate used vitamin E. In a similar vein, CoQ10 also works synergistically with Pycnogenol.

In the February 2006 issue of the Journal Evidence Based Integrative Medicine, Ronald R. Watson Ph.D. reports on the symbiotic effect of the combination of CoQ10 and Pycnogenol for promoting cardiovascular health. This synergy enhances antioxidant protection from cell damaging free radicals to help maintain a healthy capillary circulatory system. Applicant has found that the same synergy is equally applicable in the oral care field. Moreover, given the scientifically demonstrable connection between a healthy mouth and a healthy cardiovascular system, and the scientifically based knowledge that lack of attention to oral care can start a domino effect resulting in health concerns leading to significant reduction in life expectancy, the present invention can enhance total body health through its contribution to enhanced oral health.

The AC-11 works to enhance the health of the DNA and the nuclei of human cells while CoQ10 works on the inner aspect of the cells in the mitochondria. At the same time, Pycnogenol enhances the health of the inner and outer aspects of human cells. Taking all of these enhancements into account, by combining them together into a single formulation, all of the enhancements may simultaneously be achieved. The positive effects of the antioxidants included in the inventive formulation counteract the potential negative effects from the presence of free radicals. As is known, free radical damage to the cell membrane can impair a cell's ability to transport nutrients therein, resulting in cell death devoid of cell replacement. Such free radical damage to cell membranes can also impair a cell's ability to remove waste products causing premature cell death without reproduction. Free radicals can also damage the DNA of a cell, thereby causing a cell to be replaced with a mutant that fails to operate in the proper manner its parent operates. Free radicals can also damage the lysosomal sac that releases deadly enzymes that can destroy other cell components. Byproducts formed as a result of free radical reactions can interfere with cell functions. Thus, the antioxidants included in the inventive formulation help to protect the body against the negative effects of free radicals.

The inventive formulation may be applied to the patient in any one of a number of types of applications including via spray or through the use of an applicator that may be used to “paint” the formulation onto surfaces of the oral cavity. Such applicators may include an applicator tip that consists of a brush, sponge, or other porous applicator.

An Oral Breath Spray formula as outline in Example 1, was tested in a 6 week Clinical Trial at the University of Minnesota, to evaluate the effectiveness of the Smileceutical Active Ingredients vs. a Placebo in reducing bad breath (Oral Malodor), Plaque Build Up and Gingivitis.

Study Design:

Thirty subjects ranging from 18-75 years old were enrolled and randomly assigned to one of two groups (Group 1 and Group 2). Participants assigned to Group 1 were given the inventive formulation in the form of a breath spray and participants in Group 2 were given the Placebo. The breath spray had the formula listed as Example 1 in the following table. Both groups were given identical instructions on breath spray usage and were instructed to maintain their regular oral hygiene. Two visits during the course of the study (one at baseline and the other on Week 6) were also required of each subject in order to evaluate their progress. Approximately 12-14 in each treatment group successfully completed the study.

Clinical Measurement Methods:

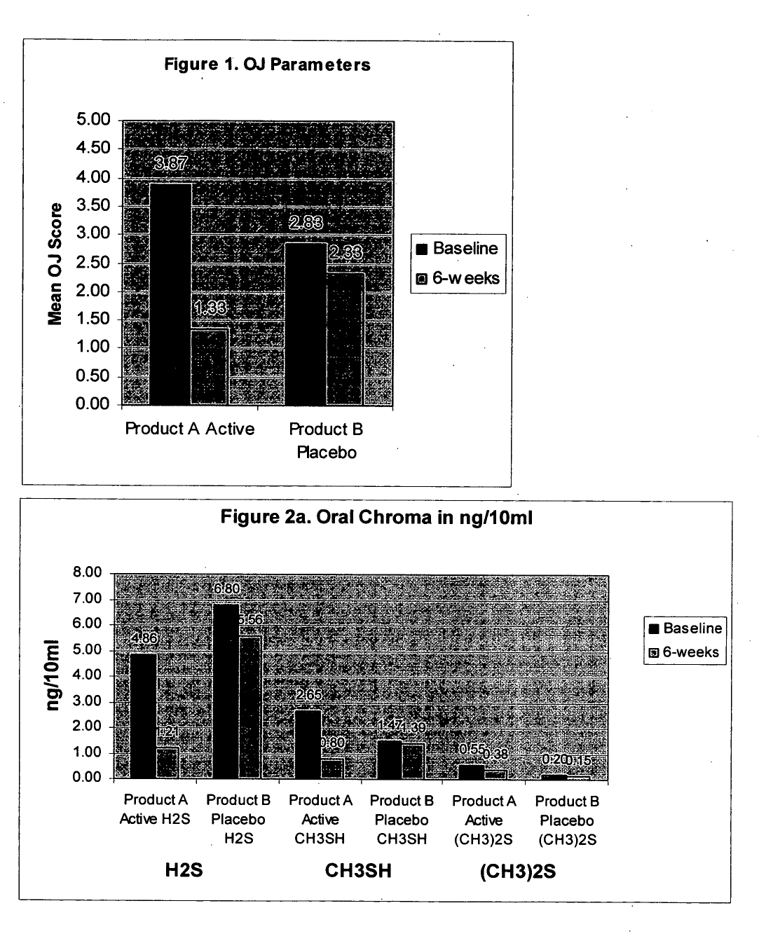

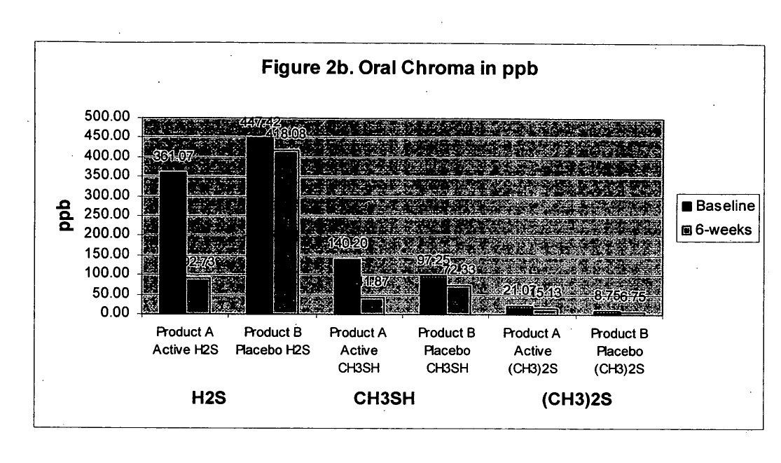

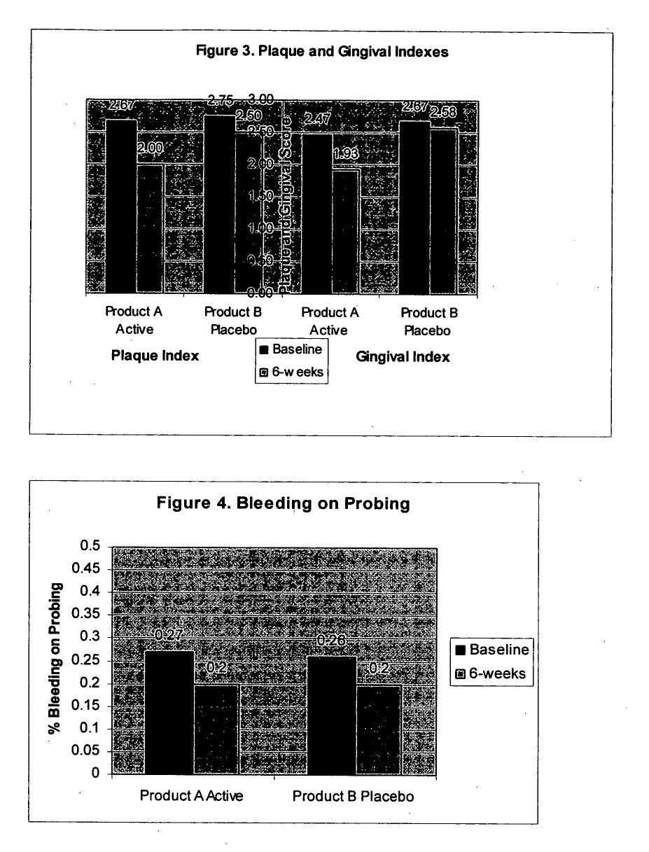

The Oral Cavity in relation to oral malodor were assessed using the following parameters:
Breath Odor assessment by 2 Independently Trained Judges (Organoleptic Judges—OJ) using a scale of 1-5 with 1=no odor and 5=strongest odor (see FIG. 1).
Oral Chroma: an instrument that measures the amount of plaque present on the tooth surface (see FIGS. 2a (ng/10 ml) and 2b (ppb)).
Gingival Index (GI) score from 0-4 with 0=no inflammation and 4=Severe Inflammation (see FIG. 3).
Bleeding Gums when probed which is a direct indication of poor gum health (see FIG. 4).
   Results:

Twenty-seven of the 30 subjects completed the 6-week clinical trial: 15 from Group 1 (breath spray) and 12 from Group 2 (Placebo). Independent statistical analyses were conducted to compare the data of each group at baseline and after 6 weeks of using the assigned product. The results show a significant reduction (65%) in Bad Breath Odor for the participants using the GO SMILE Elixir (Group 1) in comparison to Group 2 which only had a 17% reduction.

The results also show that participants using breath spray have a statistically significant improvement for the plaque and gingival index after 6 weeks of product usage. This is a direct indication of the reduction of plaque and bacteria which can cause bad breath and Gingivitis.

The following examples demonstrate the effectiveness of variations of the inventive formulation within the ranges of the constituent ingredients as disclosed herein above:

### EXAMPLES

| Ingredients | Example 1 | Example 2 | Example 3 | Example 4 |
|---|---|---|---|---|
| PART A | | | | |
| DI Water | 80.8400 | 36.3200 | 72.7000 | 49.9100 |
| Disodium EDTA [Solid] | 0.1000 | 0.1000 | 0.1000 | 0.1000 |
| Sodium Benzoate (NF or USP or FCC) | 0.1500 | 0.5000 | 0.2500 | 0.3000 |
| Potassiom Sorbate (NF or USP or FCC) | 0.2500 | 0.0000 | 0.2500 | 0.0000 |
| French Maritime Pine Bark Ext. Dry Powder [Pycnogenol] | 0.2500 | 2.0000 | 0.5000 | 5.0000 |
| AC-11 [Uncaria Tomentosa Ext.] Dry Powder [Optigenex] | 0.5000 | 1.0000 | 0.5000 | 5.0000 |
| PART B | | | | |
| Cetylpyridinium Chloride [Solid] | 0.0500 | 0.0700 | 0.1500 | 0.0800 |
| PART C | | | | |
| Sorbitol 70% (NF or USP or FCC) | 6.0000 | 20.0000 | 6.0000 | 6.0000 |
| Glycerin (NF or USP of FCC) | 3.0000 | 5.0000 | 3.0000 | 3.0000 |
| Xylitol (NF or USP of FCC) | 1.0000 | 10.0000 | 1.0000 | 5.0000 |
| Sucralose (NF or USP or FCC) | 0.7000 | 0.2000 | 0.2500 | 0.3000 |
| Propyl Gallate (NF or USP or FCC) | 0.0100 | 0.0100 | 0.0000 | 0.0100 |
| Allantoin (NF or USP of FCC) | 0.1000 | 1.0000 | 2.0000 | 5.0000 |
| Aloe Vera Powder 1S [Terry Labs] | 1.0000 | 5.0000 | 2.0000 | 10.0000 |
| Poloxamer 338 [BASF] | 0.5000 | 0.5000 | 0.5000 | 0.5000 |
| Cremophor RH40 [PEG-40 hydrogenated Castor Oil] | 1.0000 | 0.0000 | 0.5000 | 0.5000 |
| Sodium Ascorbyl Phosphate [Stay C 50] [Roche] | 0.2500 | 5.0000 | 2.0000 | 3.0000 |
| CoQ10 [ALL-Q (Coenzyme Q10) 10% CWS/S] [DSM] | 1.0000 | 10.0000 | 5.0000 | 3.0000 |
| Natural Peppermint WONF [Ungerer] FN6728 | 0.4000 | 0.4000 | 0.4000 | 0.4000 |
| Natural Orange Tangerine WONF {Ungerer] FN6727 | 2.4000 | 2.4000 | 2.4000 | 2.4000 |
| PART D | | | | |
| Citric Acid | 0.5000 | 0.0000 | 0.2500 | 0.2000 |
| Lactic Acid | 0.0000 | 0.5000 | 0.2500 | 0.3000 |
| | 100.000 | 100.000 | 100.000 | 100.000 |
| Ingredients | Example 5 | Example 6 | Example 7 | Placebo |
| PART A | | | | |
| DI Water | 77.8400 | 30.8200 | 73.2000 | 63.4900 |
| Disodium EDTA [Solid] | 0.1000 | 0.1000 | 0.1000 | 0.1000 |
| Sodium Benzoate (NF or USP or FCC) | 0.1500 | 0.5000 | 0.2500 | 0.3000 |
| Potassiom Sorbate (NF or USP or FCC) | 0.2500 | 0.0000 | 0.2500 | 0.0000 |
| French Maritime Pine Bark Ext. Dry Powder [Pycnogenol] | 0.2500 | 2.0000 | 0.5000 | 0.0000 |
| AC-11 [Uncaria Tomentosa Ext.] Dry Powder [Optigenex] | 0.5000 | 1.0000 | 0.5000 | 0.0000 |
| PART B | | | | |
| Cetylpyridinium Chloride [Solid] | 0.0500 | 0.0700 | 0.1500 | 0.0000 |
| PART C | | | | |
| Sorbitol 70% (NF or USP or FCC) | 6.0000 | 20.0000 | 6.0000 | 6.0000 |
| Glycerin (NF or USP of FCC) | 3.0000 | 5.0000 | 3.0000 | 3.0000 |
| Xylitol (NF or USP of FCC) | 1.0000 | 10.0000 | 1.0000 | 5.0000 |
| Sucralose (NF or USP or FCC) | 0.7000 | 0.2000 | 0.2500 | 0.3000 |
| Propyl Gallate (NF or USP or FCC) | 0.0100 | 0.0100 | 0.0000 | 0.0100 |
| Allantoin (NF or USP or FCC) | 0.1000 | 1.0000 | 2.0000 | 5.0000 |
| Aloe Vera Powder 1S [Terry Labs] | 1.0000 | 5.0000 | 2.0000 | 10.0000 |
| Poloxamer 338 [BASF] | 0.5000 | 0.5000 | 0.5000 | 0.5000 |
| Cremophor RH40 [PEG-40 hydrogenated Castor Oil] | 1.0000 | 0.0000 | 0.5000 | 0.5000 |
| Sodium Ascorbyl Phosphate [Stay C 50] [Roche] | 0.2500 | 5.0000 | 2.0000 | 3.0000 |
| CoQ10 [ALL-Q (Coenzyme Q10) 10% CWS/S] [DSM] | 1.0000 | 10.0000 | 5.0000 | 0.0000 |
| Natural Peppermint WONF [Ungerer] FN6728 | 0.4000 | 0.4000 | 0.4000 | 0.4000 |
| Natural Orange Tangerine WONF {Ungerer] FN6727 | 2.4000 | 2.4000 | 2.4000 | 2.4000 |
| Green Tea Extract | 2.5000 | 5.0000 | 1.0000 | 0.000 |
| Citric Acid | 1.0000 | 0.0000 | 0.0000 | 0.000 |
| Lactic Acid | 0.0000 | 1.000 | 0.0000 | 0.000 |
| | 100.000 | 100.000 | 100.000 | 100.000 |

Placebo did not contain Pycnogenol, AC-11, CPC and CoQ10 or Green Tea Extract. The formula difference was made up via water.

As such, an invention has been disclosed in terms of preferred embodiments that fulfill each and every one of the objects of the present invention as set forth hereinabove and provides a new and useful oral care formulation of great novelty and utility.

Of course, various changes, modifications and alterations in the teachings of the present invention may be contemplated by those skilled in the art without departing from the intended spirit and scope thereof.

As such, it is intended that the present invention only be limited by the terms of the appended claims.

1. An oral care formulation, comprising, by weight %:
a) 0.01-15% Pycnogenol;
b) 0.01-15% Uncaria Tomentosa extract;
c) 0.01-15% of a combination of B-complex vitamins and Vitamin C known by the trade name CoQ10;
d) 0.01-15% Stabilized Vitamin C;
e) 0-15% Green Tea Extract;
f) 0.01-20% flavorant;
g) 0.01-10% sweetener; and
h) balance water,
i) said formulation promoting enhanced oral health when applied within an oral cavity of a user.

2. The formulation of claim 1, comprising, by weight:
a) about 0.25% Pycnogenol;
b) about 0.5% Uncaria Tomentosa;
c) about 1% CoQ10;
d) about 0.25% Stabilized Vitamin C;
e) about 2.8% flavorant;
f) about 6% sweetener;
g) about 80% water.

3. The formulation of claim 1, comprising, by weight:
a) about 2% Pycnogenol;
b) about 1% Uncaria Tomentosa;
c) about 10% CoQ10;
d) about 5% Stabilized Vitamin C;
e) about 2.8% flavorant;
f) about 20% sweetener;
g) about 36% water.

4. The formulation of claim 1, comprising, by weight:
a) about 0.5% Pycnogenol;
b) about 0.5% Uncaria Tomentosa;
c) about 5% CoQ10;
d) about 2% Stabilized Vitamin C;
e) about 2.8% flavorant;
f) about 6% sweetener;
g) about 72% water.

5. The formulation of claim 1, comprising, by weight:
a) about 5% Pycnogenol;
b) about 5% Uncaria Tomentosa;
c) about 3% CoQ10;
d) about 3% Stabilized Vitamin C;
e) about 2.8% flavorant;
f) about 6% sweetener;
g) about 49% water.

6. The formulation of claim 1, comprising, by weight:
a) about 0.25% Pycnogenol;
b) about 0.5% Uncaria Tomentosa;
c) about 1% CoQ10;
d) about 0.25% Stabilized Vitamin C;
e) about 2.5% Green Tea Extract;
f) about 2.8% flavorant;
g) about 6% sweetener;
h) about 77% water.

7. The formulation of claim 1, comprising, by weight:
a) about 2% Pycnogenol;
b) about 1% Uncaria Tomentosa;
c) about 10% CoQ10;
d) about 5% Stabilized Vitamin C;
e) about 5% Green Tea Extract;
f) about 2.8% flavorant;
g) about 20% sweetener;
h) about 30% water.

8. The formulation of claim 1, comprising, by weight:
a) about 0.5% Pycnogenol;
b) about 0.5% Uncaria Tomentosa;
c) about 5% CoQ10;
d) about 2% Stabilized Vitamin C;
e) about 1% Green Tea Extract;
f) about 2.8% flavorant;
g) about 6% sweetener;
h) about 73% water.

9. A method of treating the oral cavity of a user comprising the steps of:
a) providing an oral care formulation comprising by weight percentage:
i) 0.01-15% Pycnogenol;
ii) 0.01-15% Uncaria Tomentosa extract;
iii) 0.01-15% of a combination of B-complex vitamins and Vitamin C known by the trade name CoQ10;
iv) 0.01-15% Stabilized Vitamin C;
v) 0-15% Green Tea Extract;
vi) 0.01-20% flavorant;
vii) 0.01-10% sweetener; and
viii) balance water;
b) providing an applicator and containing said oral care formulation within said applicator; and
c) using said applicator to apply said oral care formulation within said oral cavity.

10. The method of claim 9, wherein said Pycnogenol as incorporated within said formulation regenerates Vitamin C.

11. The method of claim 10, wherein Vitamin C regenerated by Pycnogenol in turn regenerates used Vitamin E.

12. The method of claim 9, wherein said Pycnogenol and CoQ10 provide a synergy that enhances antioxidant protection from cell-damaging free radicals to thereby enhance maintenance of a healthy capillary circulatory system.

13. The method of claim 9, wherein said Green Tea Extract, when included in said formulation, comprises an antioxidant delivering anti-microbial and anti-bacterial properties to relieve gingivitis, reduce plaque build-up and bad breath.

14. The method of claim 9, wherein said formulation when applied within said oral cavity reduces bad breath, gingivitis, gingival bleeding, plaque build-up and inflammation of soft and hard tissues of the oral cavity.

15. The method of claim 9, wherein said formulation when applied within said oral cavity provides antioxidant benefits to restore damaged cells.

16. The method of claim 9, wherein said formulation comprises, by weight:
a) about 0.25% Pycnogenol;
b) about 0.5% Uncaria Tomentosa;
c) about 1% CoQ10;
d) about 0.25% Stabilized Vitamin C;
e) about 2.8% flavorant;
f) about 6% sweetener;
g) about 80% water.

17. The method of claim 9, wherein said formulation comprises, by weight:
a) about 2% Pycnogenol;
b) about 1% Uncaria Tomentosa;
c) about 10% CoQ10;
d) about 5% Stabilized Vitamin C;
e) about 2.8% flavorant;
f) about 20% sweetener;
g) about 36% water.

18. The method of claim 9, wherein said formulation comprises, by weight:
a) about 0.5% Pycnogenol;
b) about 0.5% Uncaria Tomentosa;
c) about 5% CoQ10;
d) about 2% Stabilized Vitamin C;
e) about 2.8% flavorant;
f) about 6% sweetener;
g) about 72% water.

19. The method of claim 9, wherein said formulation comprises, by weight:
a) about 0.25% Pycnogenol;
b) about 0.5% Uncaria Tomentosa;
c) about 1% CoQ10;
d) about 0.25% Stabilized Vitamin C;
e) about 2.5% Green Tea Extract;
f) about 2.8% flavorant;
g) about 6% sweetener;
h) about 77% water.

20. The method of claim 9, wherein said formulation comprises, by weight:
a) about 2% Pycnogenol;
b) about 1% Uncaria Tomentosa;
c) about 10% CoQ10;
d) about 5% Stabilized Vitamin C;
e) about 5% Green Tea Extract;
f) about 2.8% flavorant;
g) about 20% sweetener;
h) about 30% water.
